(19) United States
(12) Patent Application Publication
(10) Pub. No.: US 2023/0140453 A1
(43) Pub. Date: May 4, 2023

(54) METHOD FOR CONTROLLING OBESITY BY REGULATING ACTIVITY OF TREK1

(71) Applicants: KOREA INSTITUTE OF SCIENCE AND TECHNOLOGY, Seoul (KR); KOREA FOOD RESEARCH INSTITUTE, Wanju-gun (KR)

(72) Inventors: Eun Mi HWANG, Seoul (KR); Ajung KIM, Seoul (KR); Jaekwang LEE, Jeonju-si (KR); Seoyoung JUNG, Daegu (KR)

(73) Assignees: KOREA INSTITUTE OF SCIENCE AND TECHNOLOGY, Seoul (KR); KOREA FOOD RESEARCH INSTITUTE, Wanju-gun (KR)

(21) Appl. No.: 18/052,282

(22) Filed: Nov. 3, 2022

(30) Foreign Application Priority Data
Nov. 3, 2021 (KR) 10-2021-0149971
Aug. 1, 2022 (KR) 10-2022-0095595

(51) Int. Cl. G01N 33/50

(52) U.S. Cl. CPC G01N 33/5023; G01N 2333/705

(57) ABSTRACT

The present invention relates to obesity control by regulating the expression level of TREK1.

### CROSS-REFERENCE TO RELATED APPLICATIONS

This application claims the benefit under 35 USC 119(a) of Korean Patent Application Nos. 10-2021-0149971 filed on Nov. 3, 2021, and 10-2022-0095595 filed on Aug. 1, 2022, in the Korean Intellectual Property Office, the entire disclosures of which are incorporated herein by reference for all purposes.

### REFERENCE TO AN ELECTRONIC SEQUENCE LISTING

The contents of the electronic sequence listing (“NewApp_0210920006_SequenceListing.xml”; Size is 2 KB and it was created on Nov. 2, 2022) is herein incorporated by reference in its entirety.

### TECHNICAL FIELD

The present invention relates to obesity control according to the regulation of activity of TREK1.

### BACKGROUND ART

Obesity is very common among modern people and is known as a disease that causes various lifestyle diseases. Accordingly, research on new anti-obesity substances is being actively conducted (Korean Patent No. 10-1662062), but effective treatment methods or therapeutic agents are still somewhat insufficient.

In addition, since the major anti-obesity drugs used so far act as a mechanism to suppress appetite by acting on the central nervous system or the peripheral nervous system, various side effects occur when these drugs are taken for a long period of time due to the mechanism of action, and thus are known to have low drug compliance. Therefore, there is a need for a new obesity treatment strategy with a mechanism that suppresses the division and differentiation of adipocytes rather than a method of suppressing appetite.

### DISCLOSURE

### Technical Problem

One object of the present invention provides a pharmaceutical composition for preventing or treating obesity, including a TREK1 activator as an active ingredient.

Another object of the present invention provides a method for preventing or treating obesity, including enhancing the activity of TREK1.

Still another object of the present invention provides a method for preparing an obese animal model, including weakening TREK1 activity.

Yet another object of the present invention provides an obese animal model prepared by the preparation method above.

Even another object of the present invention provides a method for screening a preventive or therapeutic agent for obesity, including:

(a) administering a candidate substance for preventing or treating obesity into an animal model; and (b) measuring the level of TREK1 activity in the animal model of step (a).

Further another object of the present invention provides a method for controlling obesity, including regulating the expression level of TREK1.

Still further another object of the present invention provides a use of TREK1 or its activator in preventing or treating obesity.

### Technical Solution

The present invention will be described in detail as follows. Meanwhile, each description and embodiment disclosed herein can be applied to other descriptions and embodiments, respectively. That is, all combinations of various elements disclosed herein fall within the scope of the present invention. Further, the scope of the present application is not limited by the specific description described below. Additionally, a number of papers and patent documents have been cited throughout the present specification. The content of the cited papers and patent documents is incorporated herein by reference in their entirety and the level of technical field to which the present invention belongs and the contents of the present invention will be described more clearly.

Additionally, those of ordinary skill in the art may be able to recognize or confirm, using only conventional experimentation, many equivalents to the particular aspects of the invention described herein. Furthermore, it is also intended that these equivalents be included in the present invention.

It is one aspect of the present invention to provide a method for controlling obesity, including regulating the expression level of TREK1.

The present inventors confirmed the phenomenon that the potassium ion conductivity is decreased and the membrane potential is depolarized in the process of differentiation into adipocytes, and it was first identified that TREK1 is specifically involved in this process. Therefore, it was confirmed that obesity was induced by promoting differentiation into adipocytes according to inhibition of TREK1, and differentiation of adipocytes was inhibited according to increase in TREK1 activity.

Accordingly, it is one aspect of the present invention to provide a use of TREK1 or its activator in treating obesity.

It is another aspect of the present invention to provide a pharmaceutical composition for preventing or treating obesity, including a TREK1 activator as an active ingredient.

As used herein, the “TREK1” is also designated as “KCNK2”, and referred to as “K2p2.1”, “TPKC1”, “hTREK-1 c”, “hTREK-1 e”, “potassium two pore domain channel subfamily K member 2”, etc. TREK1 is known to mainly transmit electrical signals in mammalian neurons or to be involved in roles such as ischemia and anesthesia. Gene or protein sequence information of TREK1 can be found in Uniprot 095069, NP_001017424, NP_001017425, NP_055032, etc., and the gene encoding TREK1 may be referred to as TREK1, KCNK2, etc.

As used herein, the “TREK1 activator” includes, without limitation, substances capable of enhancing the activity of TREK1. Such activity enhancement includes not only enhancing the activity of the protein, but also increasing the gene expression level or increasing the copy number of the gene.

Examples of substances known to increase the activity of TREK1 are disclosed in Djillani, A., Mazella, J., Heurteaux, C., and Borsotto, M. (2019) Role of TREK-1 in Health and Disease, Focus on the Central Nervous System. Front. Pharmacol. 10:379, etc.

In one embodiment, the TREK1 activator may be selected from riluzole, lithium, gabapentin, valproate, carbamazepine, ML67, ML67-33, ML335, ML402, arachidonic acid, chloroform, halothane, isoflurane, diethyl ether, fenamate, flufenamic acid, BL-1249, RNE28, GI-530159, PUFA, and derivatives thereof.

In another embodiment, the TREK1 activator may be a substance that increases the expression of a gene encoding TREK1. For example, the TREK1 activator may be an expression cassette containing the cDNA of the gene encoding TREK1, and as a specific example, it may be a vector containing the cDNA of the gene encoding TREK1.

As used herein, the “expression cassette” refers to a unit cassette which includes a promoter and a target gene and thus can express the target gene operably linked to the promoter. For the purpose of the present invention, the target gene may be a gene encoding TREK1. The expression cassette may include various factors that can assist efficient expression of the target gene, inside or outside of the expression cassette. The expression cassette may conventionally include a transcription termination signal, a ribosome-binding domain, and a translation termination signal, in addition to the promoter operably linked to the target gene, and may also include enhancer sequences, introns, translation initiation sequences and untranslated regions (UTR), etc., but is not limited thereto.

As used herein, the term “operably linked” means that the promoter sequences capable of initiating and mediating the transcription of the target gene and the gene sequence are functionally linked.

As used herein, the term “vector” refers to a DNA construct containing the polynucleotide sequence encoding the target polypeptide or protein operably linked to a suitable regulatory sequence so as to be able to express the target polypeptide or protein. For the purpose of the present invention, the target protein may be TREK1.

Once transformed into a suitable host cell, the vector may replicate or function independently from the host genome, or may integrate into genome thereof. The vector that can be used in the present invention is not particularly limited, and in one example, plasmids, viruses or bacteriophages may be used, and examples thereof include adeno-associated viruses, etc.

In any one of the above-described embodiments, the promoter included in the vector or expression cassette may be specifically expressed in fat cells, for example, adipocytes, but is not limited thereto.

As used herein the “obesity” refers to a state in which excess adipose tissues are in the body. Obesity is also caused by the differentiation of adipocytes, thereby increasing its number, and is usually accompanied by overweight. Obesity is a metabolic disease that affects the entire body, increasing the risk of diabetes, hyperlipidemia, sexual dysfunction, arthritis, and cardiovascular disease, and is also associated with occurrence of cancer in some cases. In addition, since obesity is closely related to various metabolic syndromes including non-alcoholic liver disease, the pharmaceutical composition of the present invention can also be used for the prevention and treatment of metabolic syndrome.

The pharmaceutical composition of the present invention may be administered to an obese patient, and the target obese patient may be typically determined according to the body mass index (BMI) index. In the present invention, BMI means a value obtained by dividing weight (kg) by the square of height (m), and the obese patient of the present invention is defined as an obese patient when the BMI index is 23 kg/m², 24 kg/m², 25 kg/m², 26 kg/m², 27 kg/m², 28 kg/m², 29 kg/m², 30 kg/m², 31 kg/m², 32 kg/m², 33 kg/m², 34 kg/m², 35 kg/m², 36 kg/m², 37 kg/m², 38 kg/m², 39 kg/m², or 40 kg/m² or more. The pharmaceutical composition of the present invention may exhibit effects such as weight loss, reduction in fat mass, etc. in obese patients.

As used herein, the term “prevention” refers to all activities that inhibit or delay the occurrence of obesity by administering the pharmaceutical composition, and the term “treatment” refers to all activities that improve or advantageously change the symptoms of obesity by administering the pharmaceutical composition.

The pharmaceutical composition of the present invention may further contain a pharmaceutically acceptable carrier, excipient, or diluent. The pharmaceutically acceptable carrier, excipient, or diluent may be one that is non-naturally occurring.

As used herein, the term “pharmaceutically acceptable” refers to the properties of having a sufficient amount to show a therapeutic effect and not causing adverse effects, and may be easily determined by those skilled in the art based on the factors well known in the medical field, such as the kind of disease, age, body weight, health status, sex, drug sensitivity of a patient, administration route, administration method, administration frequency, duration of treatment, a drug(s) to be mixed or administered simultaneously, etc.

The pharmaceutically acceptable excipient may include, but is not particularly limited to, for injections, a buffering agent, a preservative, an analgesic, a solubilizing agent, an isotonic agent, a stabilizing agent, etc., which may be used in combination; and for topical administrations, a base, an excipient, a lubricant, a preservative, etc.

The formulation type of the composition of the present invention may be prepared variously by being combined with a pharmaceutically acceptable carrier described above. For example, for injections, the composition may be formulated into unit-dose ampoules or multi-dose forms. The composition may also be formulated into solutions, suspensions, tablets, pills, capsules, sustained-release formulations, etc.

Examples of suitable carriers, excipients, and diluents for formulations may include lactose, dextrose, sucrose, sorbitol, mannitol, xylitol, erythritol, maltitol, starch, acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc. Additionally, the composition may further contain a filler, an anti-coagulant, a lubricant, a humectant, a fragrance, a preservative, etc.

Additionally, the composition may be formulated into a preparation of a unit dosage form suitable for the administration into a patient's body, and may be formulated into a preparation useful for protein drugs according to the conventional method in the pharmaceutical field so as to be administered by, for example, parenteral route. In one embodiment, the composition may be injected into adipose tissue.

In one embodiment, the pharmaceutical composition including the TREK1 activator of the present invention may include any pharmaceutically acceptable salt or any other compound capable of providing, directly or indirectly, a biologically active metabolite or residue thereof upon administration to an animal, including a human.

Accordingly, in any one of the above-described embodiments, the pharmaceutical composition of the present invention includes a prodrug of a TREK1 activator and pharmaceutically acceptable salts thereof, and other bioequivalents. The “prodrug” refers to a therapeutic agent prepared in an inactive form that is converted into an active form due to the action of enzymes or other substances, and/or other conditions in vivo or in its cells.

It is still another aspect of the present invention to provide a method for preventing or treating obesity, including: enhancing the activity of TREK1

In one embodiment, the enhancement of the TREK1 activity may include treatment with a TREK1 activator. The TREK1 activator is as described above.

In any one of the above-described embodiments, the enhancement of the TREK1 activity may be enhancing TREK1 activity of adipocytes.

In any one of the above-described embodiments, the enhancement of the TREK1 activity may be achieved by:

1) increasing the intracellular copy number of a polynucleotide encoding TREK1;

2) modifying the expression regulatory region of a gene encoding TREK1 with a sequence having a strong activity;

3) modifying the nucleotide sequence encoding the 5′-UTR or 3′-UTR of the gene transcript encoding TREK1;

4) modifying the amino acid sequence or the polynucleotide sequence encoding the same such that the activity of TREK1 is enhanced;

5) regulating the transcription or translation such that the activity of TREK1 is enhanced;

6) introducing a foreign polypeptide exhibiting the activity of TREK1 or a foreign polynucleotide encoding the polypeptide;

7) codon optimization of the polynucleotide encoding TREK1;

8) post-translational modification such that the activity of TREK1 is enhanced; or

9) a combination of two or more selected from above 1) to 8).

The 1) method of increasing the intracellular copy number of a polynucleotide encoding TREK1 may be achieved by introducing a vector, which is operably linked to the polynucleotide encoding the polypeptide and is able to replicate and function regardless of a host cell, into the host cell. Alternatively, the method may be achieved by introducing one copy or two copies of polynucleotides encoding the polypeptide into the chromosome of a host cell. The introduction into the chromosome may be performed by introducing a vector, which is able to insert the polynucleotide into the chromosome of a host cell, into the host cell, but is not limited thereto. The vector is as described above.

The 2) method of modifying the expression regulatory sequence of a gene encoding TREK1 may include replacing the expression regulatory region (or expression regulatory sequence) which is operably linked to the expression cassette containing the gene encoding TREK1, vector, or the gene encoding TREK1 on the chromosome with a sequence having a strong activity. For example, the method may be achieved by inducing a modification on the sequence through deletion, insertion, non-conservative or conservative substitution or a combination thereof to further enhance the activity of the expression regulatory sequence or by replacing the sequence with a sequence having a stronger activity. The expression regulatory sequence may include, but is not particularly limited to, a promoter, an operator sequence, a sequence encoding a ribosome-binding site, and a sequence regulating the termination of transcription and translation, etc. Further, the modification may include a modification of inserting an enhancer region.

The 3) method of modifying the nucleotide sequence of the 5′-UTR or 3′-UTR of the gene transcript encoding TREK1 may be achieved by, for example, modifying the UTR sequence to be optimized for expression, but is not limited thereto.

The 4) method of modifying the amino acid sequence or the polynucleotide sequence may be achieved by inducing a modification on the sequence through deletion, insertion, non-conservative or conservative substitution of the amino acid sequence of the polypeptide or the polynucleotide sequence encoding the polypeptide, or a combination thereof to enhance the activity of the polypeptide, or by replacing the sequence with an amino acid sequence or polynucleotide sequence modified to have a stronger activity, or an amino acid sequence or polynucleotide sequence modified to enhance the activity. The modification of properly inserting an intron sequence or modification to include a poly A tail may also be included in the modification of the polynucleotide sequence, but is not limited thereto.

The 5) method of regulating the transcription or translation such that the activity of TREK1 is enhanced may include attaching a sequence such as an IRES onto the RNA sequence to promote and activate translation initiation, adding a translation enhancer, adding PABP to the poly A tail end, or including a Kozak sequence optimized for expression or a modified form thereof, etc. The method may also include introducing factors that facilitate ribosome initiation and elongation, or introducing histone modifying enzymes in combination to rearrange the structure of chromatin so that transcription can occur smoothly.

The 6) method of introducing a foreign polynucleotide exhibiting the activity of TREK1 may be achieved by introducing a foreign polynucleotide encoding a polypeptide that exhibits the same/similar activity to that of the polypeptide. The foreign polynucleotide is not limited by its origin or sequence as long as it exhibits the same/similar activity as TREK1.

The 7) method of codon optimization of the polynucleotide encoding TREK1 may be achieved by codon optimization of an endogenous polynucleotide to increase the transcription or translation, or by optimizing the codons thereof such that the optimized transcription and translation of the foreign polynucleotide can be achieved within the cell. For example, among codons present in the coding sequence of TREK1, it can be modified to replace codons rarely expressed in mammals or primates with codons frequently expressed in primates.

The 8) method of post-translational modification of TREK1 may include a modification or chemical modification to enhance the activity of TREK1, for example, it may be modifying the transmembrane domain or C-terminus of TREK1 so that TREK1 is in an open form. Examples of such modifications include modifications such as phosphorylation or dephosphorylation, and one example includes dephosphorylation of the Ser333 residue at the C-terminus. However, the modification is not limited to the above-described examples.

The method of the present invention may include administering the pharmaceutical composition including the TREK1 activator in a pharmaceutically effective amount.

An appropriate total daily dose of the pharmaceutical composition may be determined within the scope of correct medical judgment by a practitioner, and the pharmaceutical composition may be administered once or several times in divided doses. However, for the purpose of the present invention, it is preferred that the specific therapeutically effective dose of the pharmaceutical composition for any particular patient be applied differently depending on the kind and degree of responses to be achieved, specific compositions including whether other agents are occasionally used therewith, the patient's age, body weight, health conditions, sex and diet, administration time, administration route, excretion rate of the composition, duration of treatment, various factors including drugs used in combination or simultaneously with the specific compositions, and similar factors well known in the medical field.

In one example, when the TREK1 activator of the present invention includes an expression cassette or vector, viral particles in any concentration necessary for effective transduction can be prepared for contact with preadipocytes in vitro or in vivo. In one example, the unit dose of the pharmaceutical composition may be determined using multiplicity of infection (MOI). MOI refers to the ratio or fold of a vector or viral genome to cells to which a nucleic acid can be delivered.

In any one of the above-described embodiments, the subject to which the method of the present invention can be applied refers to mammals including humans, rats, livestock, etc. that have or can develop the disease, but any subject that can be treated with the regulation of TREK1 activity of the present invention are included without limitation.

It is yet another aspect of the present invention to provide a method for preparing an obese animal model, including weakening TREK1 activity.

It is even another aspect of the present invention to provide an obese animal model prepared by the above method.

As used herein, the term “weakening” is a comprehensive concept including both reduced or no activity compared to its endogenous activity. The weakening may be used interchangeably with terms such as inactivation, deficiency, down-regulation, decrease, reduce, attenuation, etc.

The weakening may also include a case where the protein activity itself is decreased or removed compared to the activity of the endogenous protein due to a mutation of the polynucleotide encoding the protein; a case where the overall level of protein activity and/or concentration (expression level) in the cells/tissues is decreased due to the inhibition of expression of the gene encoding the same, or the inhibition of translation into the protein; a case where the polynucleotide is not expressed at all;

and/or a case where no protein activity is observed even when the polynucleotide is expressed. As used herein, the “endogenous activity” refers to the activity of a particular protein originally possessed by wild-type or a non-modified cells, tissues, or subjects before transformation, when a trait is altered by natural (such as introduction of an activator or inactivator) or artificial factors.

In one embodiment, the weakening of TREK1 may be achieved by:

1) deleting a part or all of the gene encoding TREK1;

2) modifying the expression regulatory region such that the expression of the gene encoding TREK1 is decreased;

3) modifying the amino acid sequence constituting the protein or the polynucleotide encoding the same such that the activity of TREK1 is removed or weakened;

4) post-translational modification such that the activity of TREK1 is removed or weakened;

5) modifying the nucleotide sequence encoding the 5′-UTR or 3′-UTR of the gene transcript encoding the protein such that the activity of TREK1 is removed or weakened;

6) introducing an antisense oligonucleotide which binds complementarily to the gene transcript encoding TREK1;

7) inhibiting the transcription or translation of the protein such that the activity of TREK1 is removed or weakened;

8) adding a promoter, which is to be reversely transcribed on the 3′ terminus of the open reading frame (ORF) of the gene sequence encoding TREK1 (Reverse transcription engineering, RTE);

9) treating a TREK1 activity inhibitor;

10) a combination of two or more selected from the methods 1) to 9) above, but is not particularly limited thereto.

For example,

the 1) method of deleting a part or all of the gene encoding TREK1 may be achieved by knocking out all of the polynucleotide encoding the endogenous TREK1 within the chromosome, or by replacing the polynucleotide with a polynucleotide having a partially deleted nucleotide, or with a marker gene. In another example, the polynucleotide sequence may be modified or deleted using gene scissors, etc. As for the gene scissors, zinc finger nuclease (ZFN), transcription activator-like effector nuclease (TALEN), clustered regularly interspaced short palindromic repeat (CRISPR), etc. are known, and these means can be used appropriately.

The 2) method of modifying the expression regulatory region (expression regulatory sequence) may be achieved by inducing a modification on the expression regulatory region (expression regulatory sequence) through deletion, insertion, non-conservative substitution or conservative substitution, or a combination thereof; or by replacing the sequence with a sequence having a weaker activity. The expression regulatory region may include a promoter, an operator sequence, a sequence encoding a ribosome-binding site, and a sequence for regulating the termination of transcription and translation, but is not limited thereto.

The 3) method of modifying the amino acid sequence or the polynucleotide sequence may be achieved by inducing a modification on the sequence through deletion, insertion, non-conservative or conservative substitution of the amino acid sequence or the polynucleotide sequence encoding the same, or a combination thereof to weaken the activity of TREK1, or by replacing the sequence with an amino acid sequence or a polynucleotide sequence modified to have a weaker activity, or an amino acid sequence or a polynucleotide sequence modified to have no activity, but are not limited thereto. For example, the TREK1 activity may be inhibited or weakened by introducing a mutation into the polynucleotide sequence to form a termination codon, but is not limited thereto.

The 4) method of post-translational modification includes modification or chemical modification such that the activity of TREK1 is weakened or removed, for example, the transmembrane domain or the C-terminus may be modified to block TREK1. Example thereof may include a modification that phosphorylates the C-terminus. Additionally, it is known that when the C-terminal 333 Ser residue of TREK1 is phosphorylated, TREK1 is inactivated.

The 5) method of modifying the nucleotide sequence encoding the 5′-UTR or 3′-UTR of the gene transcript encoding TREK1 may be achieved, for example, by substituting the nucleotide sequence with another UTR sequence having a lower expression rate than the endogenous sequence, but is not limited thereto.

The 6) method of antisense oligonucleotides that binds complementarily to the transcript of the gene encoding TREK1 include, for example, siRNA, shRNA, miRNA, aptamer, etc. Such antisense nucleotides include both coding and non-coding RNA, and may include a sequence that is partially or completely complementary to the transcript of the gene whose activity is to be weakened. Antisense nucleotides can modulate their activity by participating in actions such as transcription conflicts, DNA methylation, histone modifications, or splicing, editing, and translation of mRNA.

The 7) method of inhibiting the transcription or translation of the protein such that the activity of TREK1 is removed or weakened may include modification on the Kozak sequence to prevent translation or to inhibit translation; modification on the TREK1 gene or expression cassette containing the TREK1 gene that cause poly A tail to be degraded, and histone modifications such as methylation and deacetylation. It may also include the introduction of factors that interfere with binding of tRNA or mRNA to ribosomes or inhibit transcription.

The 8) an antisense transcript may be formed by introduction of an antisense promoter. The antisense promoter may be located upstream, downstream or in an intergenic region of the sense transcription promoter. The antisense promoter may be located in the 5′-UTR, exon, intron, or 3′-UTR of the same gene as the sense promoter, or located in an adjacent gene or intergenic region. For example, when the antisense promoter is located in the 5′-UTR, transcription can be regulated by competing with the sense promoter and elements in the 5′-UTR.

The 9) TREK1 activity inhibitor can be used without limitation in its form, such as a compound, natural extract, chemical, nucleic acid, peptide, virus, or vector containing the nucleic acid, as long as it can target and inhibit the activity of TREK1. Such activity inhibitors may be, but are not limited to, ribozymes, DNAzymes, peptide nucleic acids (PNAs), peptides or antibodies. Examples of substances known to inhibit the activity of TREK1 are disclosed in Djillani, A., Mazella, J., Heurteaux, C., and Borsotto, M. (2019) Role of TREK-1 in Health and Disease, Focus on the Central Nervous System. Front. Pharmacol. 10:379, etc.

In one embodiment, the weakening may be weakening in preadipocyte cells.

The animal model of the present invention may be any mammal, for example, rodents (e.g., mouse, rat, Mongolian gerbil), rabbits, cats, dogs, goats, sheep, pigs, horses, cows, or primates. The primate may be, for example, orangutan, gorilla, chimpanzee, or bonobos. In one example, the animal model may be any mammal other than a human, but is not limited thereto.

It is further another aspect of the present invention to provide a method for screening a preventive or therapeutic agent for obesity, including:

(a) administering a candidate substance for preventing or treating obesity into an animal model; and (b) measuring the level of TREK1 activity in the animal model of step (a).

The animal model that can be used in the screening method is not particularly limited, and may be an animal in which obesity is induced or an animal in which obesity is not induced.

The TREK1 and obesity are the same as described above.

The screening method may include selecting the candidate substance as a substance for preventing or treating obesity when the TREK1 activity level of the animal model is increased as a result of the measurement in step (b).

It is still further another aspect of the present invention to provide a method for regulating the differentiation of adipocytes, including regulating the TREK1 activity level.

The method may include inhibiting differentiation into adipocytes by increasing TREK1 activity in preadipocyte cells and/or inducing differentiation into adipocytes by weakening TREK1 activity in preadipocyte cells. In one embodiment, the method may include inhibiting differentiation into adipocytes by increasing TREK1 activity in preadipocyte cells and/or inducing differentiation into adipocytes by weakening TREK1 activity in preadipocyte cells in vitro or in vivo. The TREK1 and regulation of its activity level is the same as described above.

### Advantageous Effects

As disclosed in the present invention, not only obesity can be prevented or treated by regulating the activity of TREK1, and it can be effectively used for the preparation of obesity-induced animal models, research on regulatory mechanism for obesity in vivo, and the development of therapeutic agents.

In addition, since TREK1 of the present invention is an ion channel protein, which is a membrane protein, it does not need to penetrate into the cells, and much safer and fewer side effects can be expected compared to the method that acts on the brain.

### BRIEF DESCRIPTION OF DRAWINGS

FIGS. 1A to 1E are schematic diagrams showing a phenomenon in which the potassium conductivity of cells is significantly reduced and the cell membrane potential is depolarized in the process of differentiating the 3T3L1 cell line, which is a preadipocyte, into adipocytes (FIG. 1A).

FIGS. 2A to 2C confirmed the expression of 12 proteins with known functions among various K2P (two-pore potassium) ion channel proteins that maintain cell membrane potential, by comparing before and after inducing differentiation of the 3T3L1 cell line, which is a preadipocyte. Among these proteins, the expression of TREK1 is selectively very high and is specifically decreased in the differentiation process (FIG. 2A). Additionally, when spadin, known as a TREK1 inhibitor, was treated, the potassium conductivity of undifferentiated adipocytes was decreased and differentiation was further enhanced (FIG. 2B).

FIGS. 3A to 3F are diagrams showing that when 3T3L1 cells, which are preadipocytes, are treated with spadin, a TREK1 inhibitor, intracellular calcium concentration may increase and voltage-dependent calcium ion channels are involved in this process.

FIGS. 4A to 4E are views showing that when the primary cultured adipocytes are treated with spadin, a TREK1 inhibitor, or mRNA is inhibited by treatment with TREK1 shRNA, potassium conductivity is decreased, membrane potential is increased, and differentiation into adipocytes is significantly increased.

FIGS. 5A to 5I are diagrams showing that when a high-fat diet was performed using TREK1-knockout mice lacking TREK1 in order to confirm the importance of TREK1 for obesity at the individual level, the total body weight change in normal mice was not significant, but the weight of adipose tissue was significantly increased and the decomposition process of glucose was also significantly increased.

FIGS. 6A to 6C are diagrams showing that the size of adipocytes was significantly increased in TREK1-knockout mice and further increased by a high-fat diet.

FIG. 7 is a diagram showing that when ML402, an activator of TREK1, was treated in the differentiation process of 3T3 cells, which are preadipocytes, the differentiation into adipocytes was significantly reduced in a concentration-dependent manner.

### DETAILED DESCRIPTION OF PREFERRED EMBODIMENTS

The present invention will be described in detail by way of Examples and Experimental Examples. However, these Examples and Experimental Examples are given for illustrative purposes only, and the scope of the present invention is not intended to be limited to or by these Examples and Experimental Examples.

### Example 1. Effect of TREK1 in Differentiation Process of 3T3L1 Cells, which are Preadipocytes

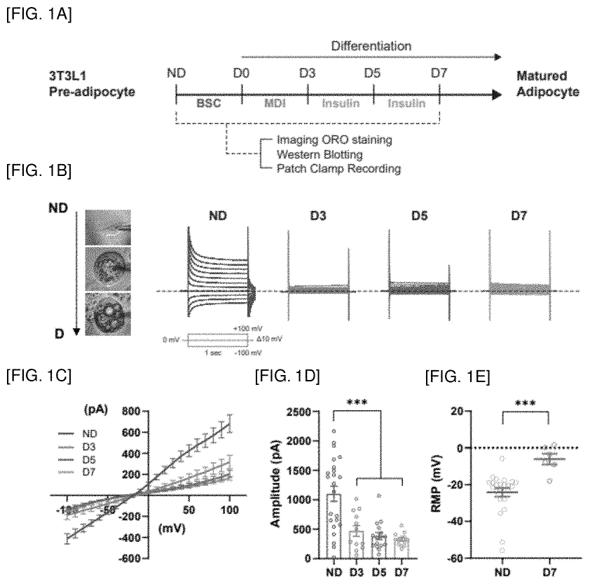

The 3T3L1 cell line, a preadipocyte cell line, was treated with MDI (Dexamethasone, IBMX, insulin) as a differentiation-inducing agent, and differentiation was induced for 7 days, and the differentiation into adipocytes was confirmed by staining with oil red 0 (FIG. 1A).

3T3-L1 cells were purchased from ATCC (American Type Culture Collection) and cultured in Dulbecco's Modified Eagle Medium (Gibco, N.Y., USA) with 10% (v/v) bovine serum (Gibco, N.Y., USA) and 1% v/v) penicillin/streptomycin (Thermo Fisher Scientific, N.J., USA). Cultures were maintained in a humidified 5% CO₂ incubator at 37° C. After reaching 80% confluence, the differentiation of 3T3L1 cells was induced by replacing the medium with DMEM containing 10% FBS plus 1 μg/mL insulin, 0.5 mM isobutyl-1-methylxanthine (IBMX), 1 μM and 2 μM Rosiglitazone for 3 days. Afterward the medium was changed with DMEM containing 10% FBS plus 1 μg/mL insulin, and the cells were further cultured for 2 to 4 days. Culture medium contained a combination of IBMX, dexamethasone (Dex), insulin, rosiglitazone, and fetal bovine serum (FBS) to differentiate preadipocyte into mature adipocytes. After differentiation, experiments were conducted at three points. Each time the medium was changed over time, the cells were differentiated with IBMX medium for 3 days, and then changed to insulin medium every 2 days thereafter. As a result of confirmation by a microscope, the cell shape of 3T3-L1 was changed over time and intracellular lipid droplets were produced.

As a result of measuring the potassium ion selective conductivity by the patch clamp technique using a cell line model in which differentiation into adipocytes was well confirmed, a sharp decrease in potassium ion conductivity was confirmed from the 3rd day, the initial stage of differentiation (FIGS. 1B to D). In addition, it was confirmed that the resting membrane potential (RMP) was highly depolarized in this process (FIG. 1E). From this result, the relevance of the K2P ion channel protein, which is known to be involved in the resting membrane potential among various potassium ion channel proteins, could be expected.

In the 3T3L1 cell line, mRNA was extracted before and after induction of differentiation to confirm the expression patterns of 12 K2P proteins whose ion channel activity have been reported. Primers reported in Supplementary Table 1 of Mi Hwang, E., Kim, E., Yarishkin, O. et al. A disulphide-linked heterodimer of TWIK-1 and TREK-1 mediates passive conductance in astrocytes. Nat. Commun. 5:3227 (2014) (https://doi.org/10.1038/ncomms4227) were used.

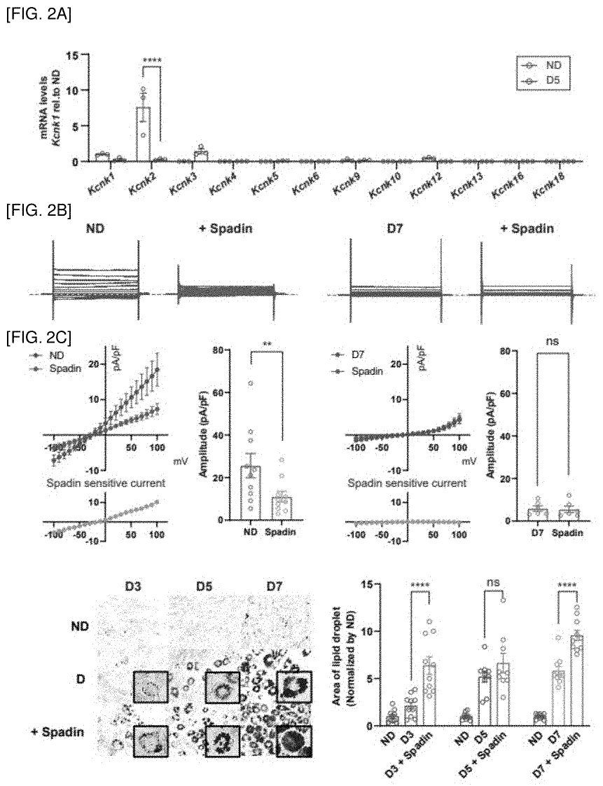

Interestingly, it was confirmed that the expression of TREK1 among the K2P proteins was selectively very high and rapidly decreased after differentiation was induced (FIG. 2A). These results were not observed in other K2P proteins, but when spadin, which is known to act very selectively on TREK1, is treated to 3T3L1 cell line, it was confirmed that the conductivity was generated by the TREK1 protein, as most of the potassium conductivity was decreased in the undifferentiated condition (FIG. 2B). In addition, it was confirmed that when spadin was treated in the process of differentiation into adipocytes, the degree of differentiation was significantly increased (FIG. 2C).

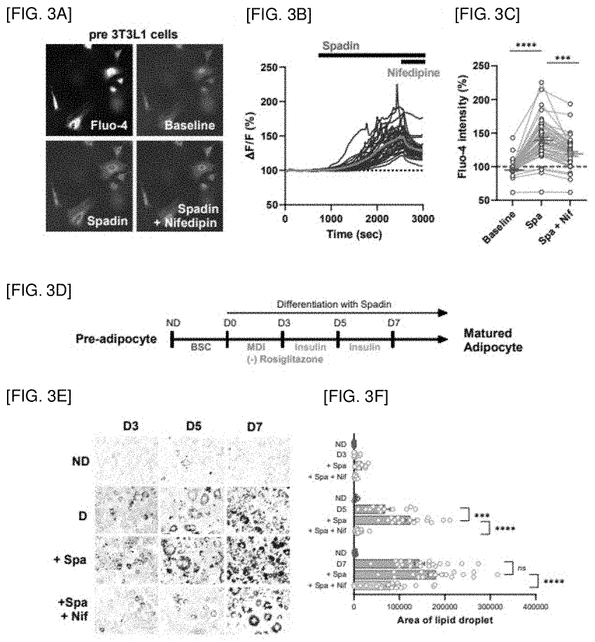

As previously known, the process of differentiation into adipocytes must be accompanied by an increase in calcium ion concentration, and as a result of treatment with spadin, a TREK1 inhibitor, and measuring the intracellular calcium ion concentration, a significant increase in calcium ions was confirmed 30 minutes after spadin treatment (FIG. 3A). This increase in calcium ions was effectively inhibited by nifedifine, a voltage-dependent calcium ion channel inhibitor (FIGS. 3B and 3C), and it was confirmed that differentiation into adipocytes was also inhibited (FIGS. 3D and 3E). In summary, the activity of TREK1, which is a potassium ion channel, was decreased in the process of differentiation into adipocytes, and as a result, the cell membrane potential was depolarized, and voltage-dependent calcium ion channels were activated, resulting in increasing intracellular calcium ion concentration and promoting the differentiation of adipocytes.

### Example 2. Effect of TREK1 in Primary Cultured Adipocytes

Since there is a possibility that the results shown in Example 1 may not reproduce the actual in vivo environment, a primary culture experiment was performed using adipocytes of mice.

The method for obtaining primary cultured adipocytes is as follows. Primary white adipocytes were isolated from inguinal WAT 6-8 week mouse (C57BL/6). The extracted WAT was washed in a 15 mL tube. The enzyme solution was mixed with collagenase Type II (Worthington Industries, Columbus, Ohio, USA) and PBS (Gibco, N.Y., USA), and the tissue was incubated in 20 mL enzyme solution in an incubator at 37° C. for 1 hour. After filtering, the tissue was added to a new 50 mL tube and centrifuged at 10,000 rpm for 10 minutes. The adipocytes floating in the solution were transferred to a new tube using a pipette. Then, the adipocytes were once more centrifuged in DMEM/F12 (Gibco, N.Y., USA) containing 10% (v/v) FBS. After transferring the fatty layer to the cell culture flask, and the flask was filled with DMEM/F12+mixture. The flask was inverted and incubated in a 37° C. CO₂ incubator, and the medium was changed after about 5 days.

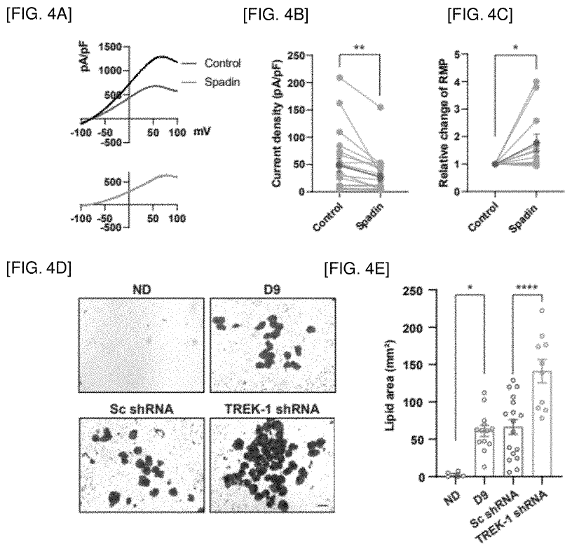

When the primary cultured adipocytes were treated with spadin (1 μM concentration), a TREK1 inhibitor, the potassium ion conductivity was significantly decreased (FIGS. 4A and 4B) as in the cell line result of Example 1, and the resting membrane potential was also increased (FIG. 4C).

In addition, differentiation was induced after treatment with TREK1 shRNA (gcgtggagatctacgacaagt, CDS 998-1018), which was verified well in previous studies, in order to confirm whether the same effect was exhibited by the reduction of TREK1 protein (see Hwang, E. M. et al. A disulphide-linked heterodimer of TWIK-1 and TREK-1 mediates passive conductance in astrocytes. Nat. Commun. 5:3227 (2014)). shRNA was used in an amount of 1 μg/μL. As a result, it was confirmed that the area of the differentiated adipocytes increased significantly compared to that treated with scrambled shRNA (FIG. 4E). This result shows that a decrease in TREK1 had an effect on the membrane potential and promoted the differentiation process in the process of differentiation of adipocytes in primary cultured mice, as in cell lines.

### Example 3. Effect on Fat Diet in TREK1-Deficient Mice

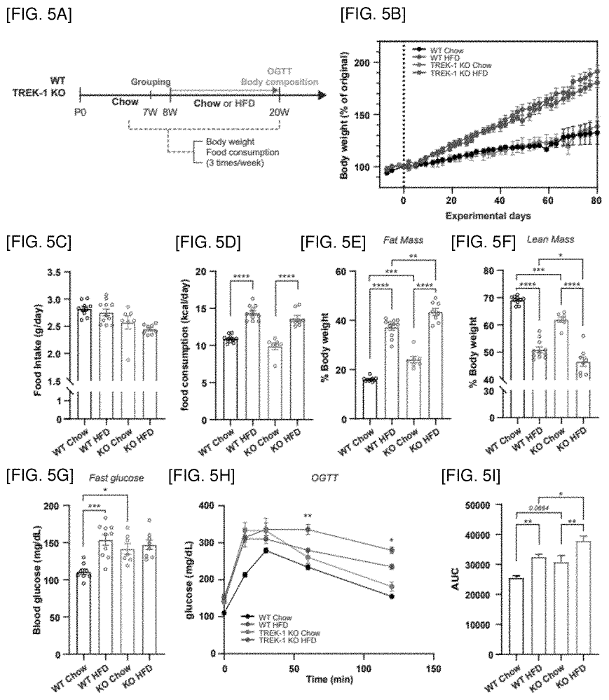

In order to confirm whether TREK1 was actually involved in the process of differentiation into adipocytes at the individual level, mice lacking only TREK1 were fed with a normal diet and a fat diet to confirm the effect on the obesity process (FIG. 5A).

TREK1-deficient mice were prepared by removing exon 4 after exon 2 including ATG using the CRISPR/Cas9 system. A feed with 10 kCal % fat content (Research Diets, D12450B) was fed to the normal diet group, and a feed with 60 kCal % fat content (Research Diets, D12492) was fed to the fat diet (HFD) group. The diet was continued for 12 weeks, and the amount of diet and weight were measured every 2 days.

Although there was no significant difference in total body weight in normal mice and TREK1-deficient mice during the 12-week diet (FIG. 5B), and there was no significant difference in intake (FIGS. 5C and 5D), it was confirmed that TREK1-deficient mice significantly increased the actual fat weight even by the normal diet (FIG. 5E). The TREK1-deficient mice increased their fat weight more than normal mice by way of the fat diet, and it was confirmed that the degradation efficiency was significantly reduced when glucose was injected after a 12-hour fast (FIGS. 5G to 5I).

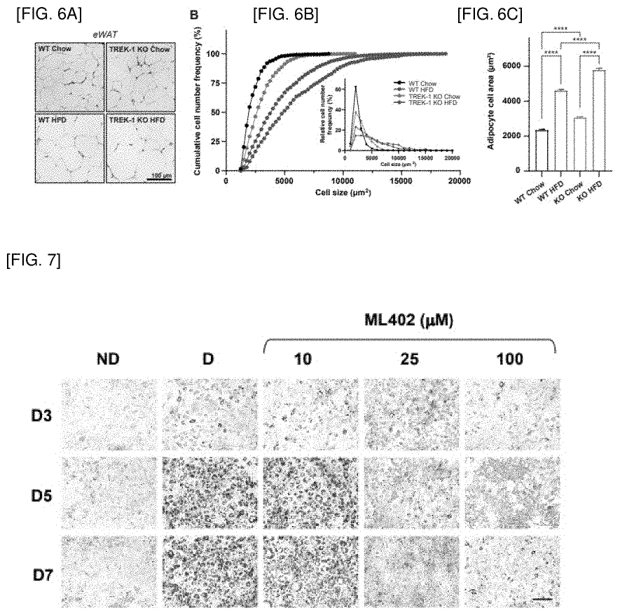

When the actual size and number of adipocytes were confirmed using a microscope, the number of adipocytes in TREK1-deficient mice was significantly increased compared to normal mice, and it was confirmed that the size was also increased (FIG. 6A). In addition, it was confirmed that the change was further increased by the fat diet (FIGS. 6B and 6C).

Based on the results, it was confirmed that inhibiting the activity of TREK1 induced obesity in both the normal diet and the fat diet groups.

### Example 4. Adipocyte Differentiation Inhibitory Effect on TREK1 Activator

Since it was confirmed through Examples 1, 2, and 3 that selective inhibition of TREK1 was essential for the differentiation process of adipocytes, it was confirmed whether treatment with an activator of TREK1 could effectively inhibit the differentiation process.

The 3T3L1 cell line, in which the same effect as in vivo and conditions was well confirmed, was treated with ML402, which has been reported to have an active effect on TREK1. Specifically, the cells were sampled on the 3rd, 5th, and 7th days by treating ML402 together from the time of differentiation at concentrations of 10 μM, 25 μM, and 100 μM, respectively. As a result of examining the effect on the differentiation process, the amount of triglycerides did not show a significant difference on the 3rd day, as compared with the control group, but decreased in a concentration-dependent manner on the 5th and 7th days, thereby confirming that the differentiation process was inhibited in a concentration-dependent manner as expected (FIG. 7).

Based on the results, it was confirmed that obesity can be suppressed by enhancing TREK1 activity.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. The scope of the present invention is therefore indicated by the appended claims rather than by the foregoing description. All changes which come within the meaning and range of equivalency of the claims are to be embraced within the scope of the present invention.

1. A method for preventing or treating obesity, comprising: enhancing the activity of TREK1.

2. The method of claim 1, wherein the enhancement of TREK1 comprises treating TREK1 activator.

3. The method of claim 1, wherein the enhancement of TREK1 is achieved by:
1) increasing the intracellular copy number of a polynucleotide encoding TREK1;
2) modifying the expression regulatory region of a gene encoding TREK1 with a sequence having a strong activity;
3) modifying the nucleotide sequence encoding the 5′-UTR or 3′-UTR of the gene transcript encoding TREK1;
4) modifying the amino acid sequence or the polynucleotide sequence encoding the same such that the activity of TREK1 is enhanced;
5) regulating the transcription or translation such that the activity of TREK1 is enhanced;
6) introducing a foreign polypeptide exhibiting the activity of TREK1 or a foreign polynucleotide encoding the polypeptide;
7) codon optimization of the polynucleotide encoding TREK1;
8) post-translational modification such that the activity of TREK1 is enhanced; or
9) a combination of two or more selected from above 1) to 8).

4. The method of claim 1, wherein the enhancement of the TREK1 activity is achieved by increasing TREK1 activity in adipocytes.

5. The method of claim 2, wherein the TREK1 activator is selected from riluzole, lithium, gabapentin, valproate, carbamazepine, ML67, ML67-33, ML335, ML402, arachidonic acid, chloroform, halothane, isoflurane, diethyl ether, fenamate, flufenamic acid, BL-1249, RNE28, GI-530159, PUFA, and derivatives thereof.

6. The method of claim 2, wherein the TREK1 activator is a vector containing cDNA

7. A method for preparing an obese animal model, comprising weakening TREK1 activity.

8. The method of claim 7, wherein the weakening is achieved by:
1) deleting a part or all of the gene encoding TREK1;
2) modifying the expression regulatory region such that the expression of the gene encoding TREK1 is decreased;
3) modifying the amino acid sequence constituting the protein or the polynucleotide encoding the same such that the activity of TREK1 is removed or weakened;
4) post-translational modification such that the activity of TREK1 is removed or weakened;
5) modifying the nucleotide sequence encoding the 5′-UTR or 3′-UTR of the gene transcript encoding the protein such that the activity of TREK1 is removed or weakened;
6) introducing an antisense oligonucleotide which binds complementarily to the gene transcript encoding TREK1;
7) inhibiting the transcription or translation of the protein such that the activity of TREK1 is removed or weakened;
8) adding a promoter, which is to be reversely transcribed on the 3′ terminus of the open reading frame (ORF) of the gene sequence encoding TREK1 (Reverse transcription engineering, RTE);
9) treating a TREK1 activity inhibitor; or
10) a combination of two or more selected from the methods 1) to 9) above.

9. An obese animal model prepared by the method of claim 7.

10. A method for screening a preventive or therapeutic agent for obesity, comprising:
(a) administering a candidate substance for preventing or treating obesity into an animal model; and (b) measuring the level of TREK1 activity in the animal model of step (a).

11. The method of claim 1, comprising selecting the candidate substance as a substance for preventing or treating obesity, when the TREK1 activity level of the animal model is increased as a result of the measurement in step (b).

12. A method for controlling obesity, comprising regulating the expression level of TREK1.


SEQUENCE LISTING (1 sequence)

SEQ ID NO: 1 - DNA, 21 bp, synthetic construct
  source 1..21: mol_type = other DNA
gcgtggagatctacgacaagt